SEQUENCE LISTING (1 sequence)

SEQ ID NO: 1 - protein, 8 aa, Artificial Sequence
  Neuropeptide FF (NPFF)
FLFQPQRF

(19) United States
(12) Patent Application Publication
(10) Pub. No.: US 2005/0130197 A1
(43) Pub. Date: Jun. 16, 2005

(54) Homogeneous fluorescence polarization assay for high throughput screening

(76) Inventors: Ernest U. Do, Seoul (KR); Su-il Kim, Yongin (KR); Jaekyoon Shin, Suwon (KR)

Correspondence Address: Joseph Hyosuk Kim, Ph.D.;JHK Law, P.O. Box 1078, La Canada, CA, 91012-1078, US

(21) Appl. No.: 10/946,302

(22) Filed: Sep. 20, 2004

Related U.S. Application Data
(60) Provisional application No. 60/504,249, filed on Sep. 19, 2003.

(30) Foreign Application Priority Data
Jul. 26, 2004 (KR) 10-2004-58396

(51) Int. Cl. C12Q 1/68; G01N 33/53

(52) U.S. Cl. 435/6; 435/7.1

(57) ABSTRACT

The present application discloses a method of determining whether an analyte binds to a target molecule in a sample comprising contacting the target molecule with a tracer molecule, wherein a bond is formed between the target molecule and the tracer molecule to form a target molecule/tracer molecule complex; contacting the target molecule/tracer molecule complex with the sample containing the analyte; and measuring fluorescence polarization of the tracer molecule over time to determine whether the analyte binds to the target molecule, wherein decreased fluorescence polarization over time indicates that the analyte binds to the target molecule, and wherein the reaction is conducted in the presence of a block copolymer surfactant.

### CROSS-REFERENCE TO RELATED APPLICATIONS

The present application claims the benefit of priority to U.S. Provisional Application 60/504,249, filed Sep. 19, 2003 and Korean Patent Application 10-2004-58396, filed Jul. 26, 2004, which are incorporated by reference herein in its entirety.

### BACKGROUND OF THE INVENTION

1. Field of the Invention

The present invention relates to the field of fluorescence polarization assay technology. The present invention also relates to a homogeneous binding assay for a receptor, using the Fluorescence Polarization (FP) format. The present invention also relates to a high throughput FP receptor binding assay. The present invention also relates to a functional assay for receptor ligands.

2. General Background and State of the Art

Traditional receptor binding assays are competitive assays in which the test compounds replace a radio-labeled molecule. These assays require either a time-consuming separation step of separating free ligands from bound ligands, and immobilization of receptors or the scintillant on a solid-phase support.

Neuropeptide FF (NPFF), Phe-Leu-Phe-Gln-Pro-Gln-Arg-Phe-NH₂ (SEQ ID NO:1) has been known to possess potent anti- and pro-opioid activity although the effects observed were dependent on the route of administration. Intracerebroventricular (ICV) administration of NPFF attenuated opiate-and stress-induced analgesia while injection of antisera against NPFF augmented morphine and stress-induced analgesia (Yang, H.-Y. T. et al., Proc. Natl. Acad. Sci. USA, (1985) 82, 7757-7761; Kivipelto, L. et al., J. Comp. Neurol., (1989) 286, 269-287; Roumy, M. et al, Eur. J. Pharm., (1998) 345, 1-11; Kavaliers, M. et al., Peptides, (1989) 10, 741-745; Malin, D. H. et al., Peptides, (1990) 11, 277-280; Lake, J. R. et al, Neurosci. Lett. (1991) 132, 29-32). NPFF possessed pro-opioid effect when it was administered into the spinal cord intrathecally, and produced a long-lasting, dose-dependent analgesia (Gouarderes, C. et al., Eur. J. Pharmacol., (1993) 237, 73-81). NPFF has been implicated in other physiological processes such as food intake, insulin release, blood pressure regulation and electrolyte balance (Panula, P. et al., Prog. Neurobiol. (1996) 48, 461-487). ICV injection of NPFF (1-5 nmol per rat) acutely reduced food intake and caused a large increase in water intake during the first 30 min after injection in food-deprived rat (Murase, T. et al., Peptides, (1996) 17, 353-354; Sunter, D. et al., Neurosci. Lett., (2001) 313, 145-148). ICV injection of NPFF inhibited arginine vasopressin (AVP) release while ICV injection of antiserum against NPFF significantly augmented plasma AVP release in conscious rats (Arima, H. et al., Endocrinology, (1996) 137, 1523-1529; Yokoi, H. et al., Peptides, (1998) 19, 393-395). NPFF elevated mean arterial blood pressure in conscious, unrestricted rats (Roth, B. L. et al., Neuropeptides, (1987) 10, 37-42; Huang, E. Y.-K. et al., Peptides, (2000) 21, 205-210). ICV injection of high doses of NPFF (8-10 μg) up-regulated its own gene expression in the nucleus of solitary tract (NTS) in the rat brain stem and elevated arterial blood pressure (Jhamandas, J. H. et al., J. Comp. Neurol., (2002) 447, 300-307). Thus, the up-regulation of NPFF may play a homeostatic role in response to specific cardiovascular challenges such as hypotension.

NPFF has been involved in interacting with other receptors in addition to opioid receptors. Administration of two NPFF receptor agonists ([D-Tyr¹, (N-Me)Phe³]NPFF) (DMe NPFF) and ([D-Tyr¹, D-Leu², D-Phe³] NPFF) (3D) produced sustained thermal and mechanical antinociception in rats. However, pretreatment with intrathecal 8-phenyltheophylline, an adenosine receptor antagonist, inhibited the antinociceptive effect of the two NPFF agonists. Administration of low dose of NPFF agonist, DMe NPFF or 3D (0.009 nmol), markedly potentiated antinociceptive actions of the adenosine receptor agonist, N6-cyclohexyladenosine, in the tail-flick and paw pressure tests (Gouarderes, C. et al., Eur. J. Pharm., (2000) 406, 391-401). The intrathecal pretreatment of NPFF reduced antinociceptive actions by α₂-adrenergic agonist dexmedetomidine in electrophysiological study (Sullivan, A. F. et al., Brain Res., (1991) 562, 327-328). NMDA-stimulated NPFF released dose-dependently even in the presence of sodium channel blocker tetrodotoxin from rat spinal cord slices, suggesting that NMDA receptors involved in the release of NPFF are mainly located on nerve terminals. The NMDA receptor antagonists, 2-amino 5-phosphonovalerate or MK-801, blocked the NMDA effect on NPFF release (Devillers, J.-P. et al., Eur. J. Pharm., (1994) 271, 185-192). Specific substance P receptor (SP-N), which was recognized by both substance P (1-11) and substance P (1-7), were involved in in vitro NPFF secretion from the rat spinal cord (Zhu, J. et al., Brain Res., 592, (1992) 326-332).

NPFF receptors have been studied in isolated membranes or in situ by employing radioligand binding assays and functional assays such as intracellular calcium mobilization assay, inhibition of forskolin-stimulated cAMP formation and phosphoinositide turnover (Elshourbagy, et al., J. Biol. Chem., (2000) 275, 25965-25971; Bonini, et al., J. Biol. Chem., (2000) 275, 39324-39331; Kotani, et al., Br. J. Pharmacol. (2001) 133, 138-144; and Liu, et al., J. Biol. Chem., (2001) 276, 36961-36969). NPFF and related peptides bound with high affinity in rodent spinal cord membranes or central nervous system (Allard, M. et al., Neuroscience, (1991) 40, 81-92; Devillers, J.-P. et al., Neuropharmacology, (1994) 33, 661-669; Allard, M. et al., Neuroscience, (1992) 49, 101-116) while opioid ligands did not compete significantly for rodent and human NPFF receptors (Devillers, J.-P. et al., Neuropharmacology, (1994) 33, 661-669; Allard, M. et al., Neuroscience, (1992) 49, 101-116; Allard, M. et al., Brain Res., (1994) 633, 127-132; Gouarderes, C. et al., Peptides, (1998) 19, 727-730; Raffa, R. B. et al., Peptides, (1994) 15, 401-404). NPFF receptors in rodents and human were coupled to a G-protein since NPFF binding was inhibited by guanine nucleotides that uncoupled receptors from G-protein (Devillers, J.-P. et al., Neuropharmacology, (1994) 33, 661-669; Payza, K. et al., J. Neurochem., (1993) 60, 1894-1899).

Two different complementary human DNA encoding G-protein coupled receptors (GPCR), NPFF1 and NPFF2 receptor, have recently been cloned (Elshourbagy, N. A. et al., J. Biol. Chem., (2000) 275, 25965-25971; Bonini, J. A. et al., J. Biol. Chem., (2000) 275, 39324-39331; Kotani, M. et al., Br. J. Pharmacol., (2001) 133, 138-144). NPFF specifically bound to NPFF1 and NPFF2 receptors with K_{d}=1.13 nM and 0.37 nM, respectively (Bonini, J. A. et al., J. Biol. Chem., (2000) 275, 39324-39331). NPFF1 and NPFF2 receptors shared about 30-35% identity of amino acid sequence with the orexin, neuropeptide Y and cholecystokinin A and prolactin-releasing hormone receptor (Bonini, J. A. et al., J. Biol. Chem., (2000) 275, 39324-39331).

There is a continuing need in the art to make more sensitive and efficient assay systems for screening compounds that are agonists or antagonists for a receptor mediated activity and for developing anti-obesity drugs.

### SUMMARY OF THE INVENTION

The present invention is directed to a homogeneous, ratiometric, binding assay using the Fluorescence Polarization (FP) format. One of the advantageous features of the inventive binding assay is that no radioisotope, separation step or solid support is needed to carry out the assay. The elimination of the separation step enhances detection of low-affinity ligands and enables a real-time, continuous readout of the binding activity in a high throughput system, such as a 384-well microplate format. The inventive assay system provides high assay window and high precision. The inventive assay system provides a specific, sensitive and reproducible methodology for functionally distinguishing agonists of a receptor from its antagonists.

In one aspect of the invention, fluorescence polarization assays may be carried out in a single well with no transfer, separation or wash steps. Thus, the invention is also directed to a high throughput drug screening method because of the advantageous speed of analysis, magnitude of displaceable signal, precision and sensitivity of various reagents.

Thus, in one aspect, the invention is directed to a method of assaying for the presence of binding between a ligand and a binding partner using a homogeneous, ratiometric, binding assay based on FP technology that can be formatted for high throughput screening. An advantage of the inventive assay system is its simple, rapid and low cost method for measuring ligand binding affinities that is amenable for high throughput screening.

In one particular aspect of the invention, the invention is directed to a method of assaying for the presence of binding between two agonists and a binding partner based on agonist-stimulated GTPγS binding that can be formatted for high throughput screening. An advantage of the inventive assay system is that the high throughput screening system provides a specific, sensitive and reproducible methodology for distinguishing agonists from antagonists when unknown ligands bind to a binding partner. Agonists of human receptors show biphasic affinity states while the antagonists give monophasic affinity states in these assays where G-protein coupled receptor activation or inactivation is desired to be assayed.

The invention is also directed to a solution system that is used in the assay system described above.

Thus, in one aspect, the invention is directed to a homogeneous assay system for determining binding of a compound to a target receptor, comprising:
(i) a tracer compound labeled with a red-shifted fluorescent dye;
(ii) a sample comprising a target GPCR;
(iii) a displacing compound;
(iv) a detergent; and
(v) at least one guanine nucleotide.

In particular, the compound may be a ligand, preferably, but not limited to a polypeptide ligand. Further, the polypeptide ligand may be any length, but preferably, may be less than about 36 amino acids, or less than about 15 amino acids, or further less than about 10 amino acids. In one aspect, the ligand may be NPFF or DMe NPFF.

The target may be any potential binding partner of the compound. Preferably, the binding between the compound and target is non-covalent. Further, the target may be a receptor, further in particular, the receptor may be a neural receptor, and further in particular, may be a NPFF receptor. Thus, in one aspect, the sample containing the receptor may be a membrane preparation.

The detergent may be an anionic, cationic, or non-ionic detergent. Preferably, the detergent may be Tween-20, poloxamer 407 series or Pluronic F-127.

The guanine nucleotides may be a guanosine di- or tri-phosphate. Preferably, the nucleotide may be guanosine 5′-diphosphate and guanosine 5′-[γ-thio]triphosphate or guanosine 5′-[β, γ-imido]triphosphate.

In one aspect, the invention is directed to a method of determining whether an analyte binds to a target molecule in a sample comprising:

(1) contacting the target molecule with a tracer molecule, wherein a bond is formed between the target molecule and the tracer molecule to form a target molecule/tracer molecule complex;

(2) contacting the target molecule/tracer molecule complex with the sample containing the analyte; and

(3) measuring fluorescence polarization of the tracer molecule over time to determine whether the analyte binds to the target molecule, wherein decreased fluorescence polarization over time indicates that the analyte binds to the target molecule, and wherein the reaction is conducted in the presence of a surfactant, which is about 2.5-4:1 ethylene oxide and propylene oxide, respectively, having a critical micelle concentration of 0.01 to 0.1% wt.

Further, the surfactant may be about 10,000 to 14,000 daltons molecular weight. In particular, the surfactant may be poloxamer 407 series or Pluronic® F-127.

Another embodiment of the invention is directed to a method of determining whether an analyte in a sample is an agonist for a receptor that is coupled to a second protein in which the second protein binds to the receptor when the receptor is activated, comprising:

(1) contacting the receptor with a tracer agonist and a fixed amount of a non-tracer known agonist in the presence of the second protein to form a mixture;

(2) contacting the mixture with the sample containing various concentrations of the analyte; and

(3) obtaining fluorescence polarization assay window of the tracer agonist at each concentration of the analyte over a set time, wherein increased assay window relative to increased concentration of the analyte indicates that the analyte is an agonist.

In this method, the contacting may take place in the presence of a surfactant, which may be a negative ionic surfactant, positive ionic surfactant, or a non-ionic surfactant. In particular, the surfactant may be non-ionic. Further in particular, the surfactant may be composed of about 2.5-4:1 ethylene oxide and propylene oxide, respectively, having a critical micelle concentration of about 0.01-0.1% wt. The surfactant may be a poloxamer 407 series or Pluronic® F-127. And in particular, the receptor may be a G-protein coupled receptor.

In the method described above, the G-protein coupled receptor may be without limitation adrenergic receptor, histamine receptor, muscarinic receptor, melanocortin receptor, neuropeptide FF receptor, epidermal growth factor receptor, neuropeptide Y receptor, dopamine receptor, cholecystokinin receptor, bombesin receptor, sphingosine 1-phosphate receptor, lysophosphatidic acid receptor, platelet-derived growth factor receptor, parathyroid hormone receptor, cannabinoid receptor, endothelin receptor, thrombin receptor, angiotensin receptor, somatostatin receptor, acetylcholine receptor, bradykinin receptor, vasopressin receptor, neurotensin receptor or opioid receptor. Preferably, the G-protein coupled receptor may be a neuropeptide receptor, and most preferably, the GPRC may be NPFF receptor or NPFF2 receptor.

Further in this method, the known agonist may be without limitation epinephrine, norepinephrine, histamine, alvameline, arecoline, cevimeline, milameline, sabcomeline, talsaclidine, tazomeline, xanomeline, melanotan-II, oxotremorine, pilocarpine, arecoline, aceclidine, propoxy-TZTP, 3-Cl-propylthio-TZTP, hexylthio-TZTP, neuropeptide FF, ([D-Tyr¹, (N-Me)Phe³]NPFF), ([D-Tyr¹, D-Leu², D-Phe³] NPFF), xylazine, medetomidine, neuropeptide Y, dopamine, cholecystokinin, bombesin, sphingosine 1-phosphate, lysophosphatidic acid, parathyroid hormone, cannabinoid, endothelin, thrombin, antiotensin, somatostatin, acetylcholine, bradykinin, vasopressin, neurotensin, or opioid.

In the methods described above, the tracer molecule may comprise a fluorescent dye. For example, the fluorescent dye may be without limitation 5-carboxytetramethylrhodamine (TAMRA). Alternatively, the tracer molecule may be, where neuropeptide FF is exemplified, fluorescein-neuropeptide FF, BODIPY-TMR-neuropeptide FF, Texas Red-neuropeptide FF, 6-TAMRA-neuropeptide FF, X-rhodamine-neuropeptide FF, Cy3-neuropeptide FF, Cy5-neuropeptide FF, or TAMRA-neuropeptide FF. In particular, the tracer molecule may be TAMRA-neuropeptide FF.

In the method described above, the receptor may be in the form of a membrane preparation. And further, a guanine nucleotide may be added to the reaction. Preferably, the guanine nucleotide may be guanosine 5′-diphosphate, guanosine 5′-[γ-thio] triphosphate, or guanosine 5′-[β, γ-imido] triphosphate.

In another aspect, the invention is directed to a method of determining whether an analyte in a sample is an antagonist for a receptor that is coupled to a second protein in which the second protein binds to the receptor when the receptor is activated, comprising:

(1) contacting the receptor with a tracer agonist and a fixed amount of a non-tracer known agonist in the presence of the second protein to form a mixture;

(2) contacting the mixture with the sample containing various concentrations of the analyte; and

(3) obtaining fluorescence polarization assay window of the tracer agonist at each concentration of the analyte over a set time, wherein substantially constant assay window relative to increased concentration of analyte indicates that the analyte is an antagonist.

In yet another aspect, the invention is directed to a composition for determining whether an analyte in a sample is an agonist or an antagonist for a receptor that is coupled to a second protein in which the second protein binds to the receptor when the receptor is activated, comprising: (i) receptor, membrane and second protein; (ii) tracer molecule; and (iii) surfactant. In the composition, the receptor may be a G-protein coupled receptor and the second protein may be a G-protein. The composition may further include a guanine nucleotide. The composition may further comprise a non-tracer known agonist for the receptor. The invention also includes a kit for determining whether an analyte in a sample is an agonist or an antagonist for a receptor that is coupled to a second protein in which the second protein binds to the receptor when the receptor is activated, comprising: (i) a container containing the receptor, membrane and second protein; (ii) a container containing tracer molecule. The kit may further. include a guanine nucleotide. The kit may also include a non-tracer agonist for the receptor.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIG. 1 shows effect of DMSO on binding of TAMRA-NPFF to human NPFF2 receptor membranes in 0.1% Pluronic F-127. ▪, bound signal; ▴, displaced signal in the presence of 1 μM neuropeptide FF. TAMRA-NPFF concentration used is 0.5 nM. Assay was performed in 384-well plates with 40 μl volume per well. Error bars represent ±1 standard deviation unit of six separate determinations.

FIG. 2 shows effect of Tween 20 on binding of TAMRA-NPFF to human NPFF2 receptor membranes. ▪, bound signal; Δ, displaced signal in the presence of 1 μM neuropeptide FF. TAMRA-NPFF concentration used is 0.5 nM. Error bars represent ±1 standard deviation unit of six separate determinations.

FIG. 3 shows concentration effect of Pluronic F-127 on binding of TAMRA-NPFF to human NPFF2 receptor membranes. ▪, bound signal; ▴, displaced signal in the presence of 1 μM neuropeptide FF. TAMRA-NPFF concentration used is 0.5 nM. Assay was performed in 384-well plates with 40 μl volume per well. Error bars represent ±1 standard deviation unit of six separate determinations.

FIG. 4 shows effect of tartrazine on bound (▪) and fully displaced (▴) TAMRA-NPFF to human NPFF2 receptor membranes. TAMRA-NPFF concentration used is 0.5 nM. Error bars represent ±1 standard deviation unit of six separate determinations.

FIG. 5 shows effect of Chicago Sky Blue 6B on bound (▪) and fully displaced (▴) TAMRA-NPFF to human NPFF2 receptor membranes. TAMRA-NPFF concentration used is 0.5 nM. Error bars represent ±1 standard deviation unit of six separate determinations.

FIG. 6 shows displacement of TAMRA-NPFF from human NPFF2 receptor by agonists, NPFF and PQRFamide. TAMRA-NPFF (0.5 nM) and 3.6 ug NPFF2 receptor membrane protein were incubated and NPFF or PQRFamide at different concentrations was added at time zero. Six replicate curves were run within a plate. Data points show mean±SEM.

FIG. 7 shows binding stability of TAMRA-NPFF to human NPFF2 receptor membranes. TAMRA-NPFF (0.5 nM) and 3.6 ug NPFF2 receptor membrane protein were incubated and [D-Tyr¹, (NMe)Phe³]NPFF (DMe NPFF) at different concentrations was added at time zero. Data points show mean±SEM of six separate determinations. ▪, bound signal; ▴, displaced signal; ▾, mP=the difference between the bound and displaced signal; Z′=1−[(3*σ_{bound})+(3*σ_{displaced})/|(mP_{bound}−mP_{displaced})|].

FIG. 8 shows displacement of TAMRA-NPFF from human NPFF2 receptor by ligands. TAMRA-NPFF (0.5 nM) and 3.6 ug NPFF2 receptor membrane protein were incubated, and DMe NPFF, frog pancreatic polypeptide (frog PP) or BIBP 3226 at different concentrations was added at time zero. Data points show mean±SEM of six separate determinations.

FIG. 9 shows the optimization of NPFF2 receptor binding by guanine nucleotides. 1 μM DMe NPFF, TAMRA-NPFF (0.5 nM) and 3.6 ug NPFF2 receptor membrane protein in 10 μl assay buffer containing 3 mM MgCl₂ were added sequentially. After 15 min, 10 μl of the binding buffer with 3 μM GDP and 5 μM of GTPγS was added. The reaction was incubated at room temperature for 60 min. Error bars represent ±1 standard deviation unit of six separate determinations.

FIG. 10 shows the effect of agonists on binding of GTPγS to human NPFF2 receptor membranes. The concentration of one agonist was fixed at 2˜3×IC50 value to give an assay window of 40-70 mP. The concentrations of the other agonist increased from 0.0019 nM to 9.0 μM per well in these experiments. The data represent the observed signals after 30 min incubation upon the addition of 5 μM GTPγS solution. Error bars represent ±1 standard deviation unit of six separate determinations.

FIG. 11 shows the effect of antagonist BIBP 3226 on binding of GTPγS to human NPFF2 receptor membranes. The concentration of one agonist was fixed at 2˜3×IC50 value to give an assay window of 40-60 mP. The concentrations of the antagonist increased from 0.017 nM to 9.0 μM per well in these experiments. The data represent the observed signals after 60 min incubation upon the addition of 5 μM GTPγS solution. Error bars represent ±1 standard deviation unit of six separate determinations.

### DETAILED DESCRIPTION OF THE PREFERRED EMBODIMENTS

In the present application, “a” and “an” are used to refer to both single and a plurality of objects.

As used herein, “target molecule” is the molecule to which the tracer and the displacer molecule binds. An example of a target molecule is an enzyme or a receptor.

Surfactant

The present invention is directed to a detergent or surfactant composition for carrying out the inventive fluorescence polarization assays, which provides a wide assay window for optimum conditions to carry out high throughput FP assays. In particular, nonionic detergents for solubilizing large dye-target complexes may be used in the practice of the invention. In particular, poloxamer or Pluronic® series of surfactants are desirable to be used in the invention. Poloxamer 407 series polymers are preferred as are Pluronic® F-127.

Preferred surfactants may include block copolymers such as poloxamer or Pluronic® type consisting of ethylene oxide (EO) and propylene oxide (PO) blocks arranged in a basic A-B-A structure: EOₓ-PO_{y}-EOₓ. Due to their amphiphilic nature, the block copolymers are able to self-assemble into micelles in aqueous solutions above critical micelle concentration (CMC). Below the CMC, the copolymers exist in solution in the form of a molecular dispersion of individual block copolymer molecules. Variations in the number of hydrophilic EO units (x) and lipophilic PO units (y) result in copolymers with different molecular mass and distinct hydrophilic-lipophilic balance. Copolymers with a short hydrophilic poly-EO block and/or an extended lipophilic poly-PO block (such as Pluronic L121 and L101) are highly lipophilic and are characterized by a relatively low CMC. In contrast, copolymers with an extended hydrophilic poly-EO block or/and short lipophilic poly-PO block (such as Pluronic F108 and F88) are hydrophilic and are characterized by relatively high CMC. Pluronic compositions such as P85 or P103 are intermediate in their lipophilicity and have a CMC value that falls between the two extremes identified above.

In particular, the inventive assay is directed to using a copolymer such as Pluronic® surfactant that has a combination of EO and PO units in a ratio of between about 2.5-4:1, respectively. Preferably, the ratio is about 3 to 1. Further, the molecular weight of the surfactant may be between about 10,000 to 14,000 daltons. Preferably, the range may be between about 11,000 to 13,000 daltons. More preferably, the molecular weight may be about 12,600 daltons. Moreover, the range of CMC of the surfactant polymer may be in the range of about 0.01 to 0.1% wt. Preferably, the CMC may be 0.02 to 0.09% wt. More preferably, the CMC may be 0.03 to 0.09% wt. Most preferably, the CMC may be about 0.08% wt. In the most preferred embodiment, the surfactant polymer may be that which has the same or substantially the same structural and functional characteristics as Pluronic® F-127.

Agonist/Antagonist Assay

In the inventive agonist determining assay for a ligand-receptor, the receptor is typically coupled to a second protein, typically a guanyl nucleotide-binding protein (G-protein). The second protein is normally not physically attached to the receptor in the absence of an agonist. However, when an agonist binds to the receptor the second protein also physically attaches to the receptor.

In the agonist detection assay of the invention, the reactants may include: (1) a receptor and second protein, (2) a fixed concentration of a non-tracer known agonist, (3) a tracer agonist of a fixed concentration, and (4) an analyte, which may be a peptide or chemical agent. The reaction may occur under binding assay conditions. The added concentration of the analyte may be varied. Polarization measurements may be taken at 0 reaction time and at a set reaction time for each concentration of the added analyte. When a graph is plotted with the concentration of the analyte on the x-axis and the change in the polarization measurement between the bound and displaced tracer (assay window) on the y-axis, a biphasic curve is seen relative to increasing amounts of the added analyte if the analyte is an agonist. A monophasic curve is seen if the analyte is an antagonist.

An agonist/receptor binding causes a biphasic effect in the presence of the second protein and a fixed concentration of the known agonist, and a fixed concentration of the known tracer agonist. While not being bound by theory, it is believed that the analyte of varying concentration further competes with the receptor-tracer complex and displaces the bound tracer because the concentration of fixed agonist (2-3×IC₅₀) has been set not to displace fully the bound tracer. Thus, as the amount of the analyte is increased, and as more analyte is bound, there is additional change in polarization measurement due to the release of the tracer from the receptor-tracer complex, saturating the specific (high affinity) binding sites. However, if the analyte is an antagonist to the receptor, the increased amount of the analyte would not readily compete with the bound tracer and the fixed agonist. Thus, an antagonist does not displace the bound tracer and the fixed agonist from the receptor-tracer complex and the receptor-agonist complex due to the higher affinity of these complexes. Therefore, the receptor binds the non-tracer agonist and the tracer agonist with greater specificity and higher affinity than the antagonist. In the instance where the receptor is GPCR and the second protein is a G-protein, the greater specificity of the agonist for the receptor may be facilitated in the presence of GDP and GTPγS. This phenomenon occurs because the free and bound tracer molecules are present simultaneously in the same well in the fluorescence polarization assay.

In one aspect of the invention, the receptor may be any receptor so long as a second protein is able to bind to the receptor upon activation of the receptor by the agonist. The receptor may be an integral membrane protein. In particular, the receptor may be a G-protein coupled receptor (GPCR). Moreover, the second protein may be a G-protein.

An integral membrane protein is a protein molecule (or assembly of proteins) that in most cases spans the biological membrane with which it is associated or which, in any case, is sufficiently embedded in the membrane to remain with it during the initial steps of biochemical purification. The integral membrane proteins perform many fundamental cellular functions. For example, they can be receptors, channels or enzymes.

A G-protein-coupled receptor (GPCR) is one of a class of integral membrane proteins belonging to the “7TM” superfamily of transmembrane receptors, examples being receptors of the olfactory epithelium that bind odorants, and receptors of the neurotransmitter serotonin in the mammalian brain. Upon ligand binding, these receptors activate G proteins. Further, the extracellular parts of the receptor can be glycosylated.

While in other types of receptors that have been studied ligands bind externally to the membrane, the ligands of G-protein-coupled receptors typically bind within the transmembrane domain. The transduction of the signal through the membrane by the receptor is not completely understood. It is believed that a receptor molecule exists in a conformational equilibrium between active and inactive states. The binding of ligands to the receptor may shift the equilibrium. Three types of ligands exist: agonists are ligands which shift the equilibrium in favour of active states; inverse agonists are ligands which shift the equilibrium in favour of inactive states; and neutral antagonists are ligands which do not affect the equilibrium. If a receptor in an active state encounters a G protein, it may activate it.

G-proteins belong to the larger grouping of GTPases. “G-protein” usually refers to the membrane-associated heterotrimeric G-proteins, sometimes referred to as the “large” G-proteins. These proteins are activated by G-protein coupled receptors and are made up of alpha (α), beta (β) and gamma (γ) subunits. There are also “small” G proteins or small GTPases like ras that are monomeric and not membrane-associated, but also bind GTP and GDP and are involved in signal transduction.

Receptor activated G-proteins are bound to the inside surface of the cell membrane. They consist of the G_{β} and the tightly associated G_{βγ} subunits. When a ligand activates the G-protein coupled receptor, the G-protein binds to the receptor, releases its bound GDP from the G_{α} subunit, and binds a new molecule of GTP. This exchange triggers the dissociation of the G_{α} subunit, the G_{βγ} dimer, and the receptor. Both, G_{α}-GTP and G_{βγ}, can then activate different ‘signalling cascades’ (or ‘second messenger pathways’) and effector proteins, while the receptor is able to activate the next G-protein. The G_{α} subunit will eventually hydrolize the attached GTP to GDP by its inherent enzymatic activity, allowing it to reassociate with G_{βγ} and starting a new cycle.

A G-protein coupled receptor may include without limitation, adrenergic receptor, histamine receptor, muscarinic receptor, melanocortin receptor, neuropeptide FF receptor, epidermal growth factor receptor, neuropeptide Y receptor, dopamine receptor, cholecystokinin receptor, bombesin receptor, sphingosine 1-phosphate receptor, lysophosphatidic acid receptor, platelet-derived growth factor receptor, parathyroid hormone receptor, cannabinoid receptor, endothelin receptor, thrombin receptor, angiotensin receptor, somatostatin receptor, acetylcholine receptor, bradykinin receptor, vasopressin receptor, neurotensin receptor and opioid receptor.

High Throughput Assay

Detecting or quantitating analyte-specific binding events is also important in high-throughput methods being developed for combinatorial library screening. In a typical method, a large library of possible effector molecules (analytes) is either synthesized or purified from natural sources. The library members are then screened for effector activity by their ability to bind to a selected receptor. The approach has the potential to identify, for example, new oligopeptide, glycopeptide, or any small molecule capable of interacting with a selected pharmacological target, such as a membrane bound receptor or cellular enzyme.

The inventive high-throughput screening methods typically employ simple analyte displacement assays to detect and quantitate analyte binding to a receptor. The inventive displacement assays have the advantage of high sensitivity, e.g., where the displaced or tracer analyte is labeled with a fluorescent dye, in a binding buffer containing a surfactant containing ethylene oxide and propylene oxide in a 2.5-4:1 ratio. The assays also allow for the determination of analyte-receptor binding affinity based on competitive displacement of a binding agent whose binding affinity to the target receptor is known. In one aspect of the invention, high throughput assays can be conducted in multi-well plates.

Tracer Compound

The tracer dye compound may be without limitation fluorescein, BODIPY-TMR, Texas Red, 6-TAMRA, X-rhodamine, Cy3, Cy5 or 5-TAMRA.

Kit

An inventive fluorescence polarization assay kit for detecting the presence of a ligand that binds to a target molecule may include the following components: (i) at least one container that contains a tracer compound labeled with a red-shifted fluorescent dye that binds to a target molecule such as a membrane preparation of GPCR; (ii) a container that contains the target molecule; and (iii) a container that contains a surfactant having 2.5-4:1 ethylene oxide, propylene oxide unit ratio and a CMC concentration of about 0.01 to 0.1% wt, which may preferably be Pluronic® F-127 or poloxamer 407 series of polymers.

It is understood that all or part of the above components may be combined and stored in a single container so long as by combining them, there is no degradation of the chemicals so as to provide accurate intended results at the time of assay.

The kit may further comprise a displacing compound, and may contain control displacer compounds to test the kit for effectiveness.

In a fluorescence polarization assay for agonists or antagonists to GPCR, a further component of at least one guanine nucleotide may be included.

Further optionally included with the kit may be a device such as a tube, dropper, pipette, micropipette, and so forth for transferring at least a measured or predetermined amount or portion of the reaction sample to conduct the polarization readings.

The container for housing the reagents and solutions may include but is not limited to an ampoule, vial, bottle and the like. A container such as an ampoule may be hermetically sealed so as to be broken by breaking the seal, or the container may be sealed and resealed upon opening such as by a twist cap.

Method of Detecting Ligand for a Target Molecule in a Sample

The present invention is directed to a method of detecting the presence and relative quantity of a ligand for a target molecule in a test sample comprising: (i) making a reagent solution containing a tracer compound, target molecule, and displacer compound, in a surfactant solution; (ii) causing a displacement reaction to occur over a set time period such as 30, 60, 90 minutes, 2 hours or longer and taking a polarization time point measurement over a course of the reaction; and (iii) determining whether there is a decrease in polarization of the tracer compound over time, wherein a decrease in polarization indicates the presence of the ligand for the target molecule in the test sample.

In the process of making the reagent solution according to step (i) above, it is understood that in one aspect, the surfactant has properties that are similar to Pluronic® F-127, such as a block copolymer in which the ethylene oxide and propylene oxide amounts are present in a range of about 2.5-4:1. The CMC may be about 0.01 to about 0.10% wt, wherein the most preferred CMC may be about 0.08% wt. Independently, the surfactant may have a molecular weight of about 10,000 to about 14,000 daltons.

The amount of the reagents to be mixed may be varied and may be optimized according to the level of specificity of the displacer compound for the target molecule and the total volume of the reaction.

The various reagents described above may be preferably stored separately in individual, sealed test-size ampoules or vials of conventional medical solution type or in any other type of packaged or unpackaged container. Alternatively, the reagents may be mixed together and stored in individual, sealed test-size ampoules or vials of conventional medical solution type or in any other type of packaged or unpackaged container.

Method of Detecting Presence of Agonist or Antagonist to a Receptor in a Sample

The present invention is directed to a method of detecting the presence of an agonist or an antagonist to a receptor in a test sample comprising: (i) making a reagent solution containing a tracer compound that may itself be an agonist to the receptor, a receptor membrane preparation which contains a coupled second protein that may bind to the receptor when the receptor is activated, and an analyte, which may be a potential agonist, antagonist or a non-reactant, in a surfactant solution; (ii) causing a displacement reaction to occur for several different concentrations of the analyte over a set time period such as 30, 60, 90 minutes, 2 hours or longer and taking a polarization time point measurement over a course of the reaction; and (iii) determining the polarization assay window by calculating a change in polarization from the beginning to the end of the reaction at each concentration of the analyte added, wherein an increase in polarization assay window of the tracer compound relative to increased amount of the displacer compound added indicates that the analyte is an agonist to the receptor. However, when there is no increase in the polarization assay window over a concentration range of the analyte tested, then the analyte may be an antagonist.

In the process of making the reagent solution according to step (i) above, it is understood that in one aspect, the surfactant may have properties that are similar to Pluronic® F-127, such as a block copolymer in which the ethylene oxide and propylene oxide amounts are present in a range of about 2.5-4:1. The CMC of the surfactant may be about 0.01 to about 0.1% wt, wherein the most preferred CMC is about 0.08% wt. Independently, the surfactant may have a molecular weight of about 10,000 to about 14,000 daltons.

The amount of the reagents to be mixed may be varied and may be optimized according to the level of specificity of the displacer for the target molecule and the total volume of the reaction.

The various reagents described above may be preferably stored separately in individual, sealed test-size ampoules or vials of conventional medical solution type or in any other type of packaged or unpackaged container. Alternatively, the reagents may be mixed together and stored in individual, sealed test-size ampoules or vials of conventional medical solution type or in any other type of packaged or unpackaged container.

It is also generally contemplated that GPCRs and G-proteins are part of the cell's second messenger signal transduction system. Interestingly, diseases such as diabetes, alcoholism, obesity and certain forms of pituitary cancer, among many others, are thought to have some root in the malfunction of GPCRs and G-proteins, and thus a fundamental understanding of their function, signaling pathways and protein interactions may lead to eventual treatments and possibly the creation of various preventative approaches. Therefore, it should be especially appreciated that the methods and compositions according to the inventive subject matter may also be useful in detecting and/or confirming and treating abnormal signal transduction states, including diabetes and obesity.

Neuropeptide FF Receptor

Radioligand binding to neuropeptide FF receptors is a common assay format currently used in the receptor binding assays (Allard, M. et al., Neuroscience, (1991) 40, 81-92; Devillers, J.-P. et al., Neuropharmacology, (1994) 33, 661-669; Allard, M. et al., Neuroscience, (1992) 49, 101-116; Payza, K. et al., J. Neurochem., (1993) 60, 1894-1899; Allard, M. et al., Brain Res., (1994) 633, 127-132; Gouarderes, C. et al., Peptides, (1998) 19, 727-730; Raffa, R. B. et al., Peptides, (1994) 15, 401-404; Elshourbagy, N. A. et al., J. Biol. Chem., (2000) 275, 25965-25971; Bonini, J. A. et al., J. Biol. Chem., (2000) 275, 39324-39331; Kotani, M. et al., Br. J Pharmacol., (2001) 133, 138-144; Liu, Q. et al., J. Biol. Chem., (2001) 276, 36961-36969). This is the first report on the homogeneous, ratiometric NPFF receptor binding assay based on FP technology that can be formatted for HTS. The assay employs the use of commercially available human recombinant NPFF2 receptor membrane preparation, an assay buffer system containing 0.1% Pluronic F-127, and a red-shifted, neutral fluorophore, TAMRA-NPFF. Pluronic F-127 is a non-ionic surfactant that consists of block polymers of ethylene oxide and propylene oxide and has been used in receptor binding assays employing optical biosensor technology (Hoffman, T. L. et al., Proc. Natl. Acad. Sci. USA 97 (2000) 11215-11220). It has an average molecular weight of 12,600 and greater ability to decrease molecular rotation of fluorescent ligands than Tween 20 (M.W.˜1,228) or PEG (M.W.˜3,350) due to its higher molecular volume and lower critical micelle concentration, 0.08 wt.-% (Nace, V. M., In Nonionic surfactants: Polyoxyalkylene block copolymers. (1996) Marcel Dekker, New York; Kang, G. D. et al., Macromol Rapid Commun., (2000) 21, 788-791). 0.1% Pluronic system was less sensitive to receptor expression level of 1 pmol/mg protein compared to 0.1% Tween 20 or 4% PEG system, and consistently has given the widest assay windows and the highest precision among the three detergents we have tested.

TAMRA-NPFF and BODIPY® TMR-NPFF gave wide assay windows. However, TAMRA-NPFF appeared to give more reproducible standard deviations than BODIPY® TMR-NPFF in the FP assays of NPFF2 receptor (unpublished work). As demonstrated in the examples of tartrazine and Chicago Sky Blue 6B, when TAMRA-NPFF is used, the interferences of colored compounds are substantially reduced. This suggests that assays conducted with TAMRA are less susceptible to false-negative results relative to common fluorescein-labeled ligands reported for similar experiments in other receptor systems using a fluorescein (Banks, P. et al., J Biomol Screen, (2000) 5, 329-334; Banks, P. et al., J Biomol Screen, (2000) 5, 159-167).

It is evident that the rank order of potency obtained from FP measurements agrees well with the conventional radioisotope filtration data reported in the literature: DMe NPFF>NPFF>frog PP (Rana temporaria pancreatic polypeptide)>PQRFamide>BIBP 3226 (Allard, M. et al., Neuroscience, (1991) 40, 81-92; Gouarderes, C. et al., Peptides, (1998) 19, 727-730; Bonini, J. A. et al., J. Biol. Chem., (2000) 275, 39324-39331; Kotani, M. et al., Br. J. Pharmacol., (2001) 133, 138-144; Mollereau, C. et al., Eur. J. Pharmacol., (2002) 451, 245-256). The notable exception to this is frog PP. The inhibitory potency of frog PP, an NPFF2-selective agonist, measured by FP binding assay, is greater than that of PQRFamide. This rank order of potency is reversed when determined by radioligand binding assay in the literature (Bonini, J. A. et al., J. Biol. Chem., (2000) 275, 39324-39331). Frog PP, a 36-mer, at its low concentrations appeared to displace TAMRA-NPFF in our assay conditions due to the more favorable surfactant and FP effect of 0.1% Pluronic, resulting in appropriate conformation for ligand binding (Nace, V. M., In Nonionic surfactants: Polyoxyalkylene block copolymers. (1996) Marcel Dekker, New York; Kang, G. D. et al., Macromol. Rapid Commun., (2000) 21, 788-791). Therefore, it gave a lower Kᵢ value than PQRFamide. The potency order of frog PP is maintained relative to BIBP 3226 reported by other researchers (Bonini, J. A. et al., J. Biol. Chem., (2000) 275, 39324-39331; Mollereau, C. et al., Eur. J. Pharmacol., (2002) 451, 245-256).

Bradykinin, capsaicin (vanilloid receptor agonist), phenoxybenzamine (calmodulin antagonist) and the five tested adrenergic ligands showed little or no affinity to the TAMRA-NPFF binding site. These negative binding results suggest that it is unlikely that these other receptor ligands bind to NPFF receptors. The potential interactions between the tested ligands and NPFF receptors in vivo may be at the level of second messenger systems.

The FP NPFF receptor binding assay is fast and does not require radiolabeled compounds, transfer, separation of bound and free tracer, or wash steps. The FP assay offers the following advantages over the radioligand binding assay: a direct measurement of the bound and displaced ratio of the tracer, and of the ligand- receptor binding kinetics, simple preparations before and during the assays, easy miniaturization to HTS format using a 384-well microplate, a longer shelf life of TAMRA-NPFF than the ¹²⁵I-labeled NPFF, no pre-equilibrium of reagents, and finally less cost per compound screening. One area of improvement in the current FP assay is to increase the low expression level of human recombinant NPFF2 receptor, 1 pmol/mg protein, which is the lower limit of successful FP binding assay (Lee, P. H. et al., J Biomol Screen, (2000) 5, 415-419). The assay performance is excellent for HTS with Z′ values greater than 0.5 for both agonist and antagonist binding assays and can tolerate up to 2% DMSO and up to 2 μM colored compound. Thus, the FP binding assay of NPFF2 receptor appears to be a sensitive and cost-effective HTS format. The assay is useful for screening potential anti-obesity drugs. Agonists of human receptors show biphasic affinity states while the antagonists give monophasic affinity states in the in vitro functional assays. Other FP binding assays of G protein-coupled receptors (GPCRs) can be formatted by use of 0.1% Pluronic F-127 buffer system, TAMRA-labeled ligand and a receptor membrane quantity corresponding to radiobinding assay. Therefore, this method will be widely applicable to recombinant GPCRs.

We have developed a sensitive and specific binding assay for human NPFF2 receptor. Since Ki values calculated from FP data agreed closely with those obtained from radioligand binding assays, contributions from non-specifically bound ligand did not significantly affect results. The salient features of the current FP assay include an assay buffer system containing Pluronic F-127, a red-shifted fluorescent dye, TAMRA-NPFF, the readily available human NPFF2 receptor membrane preparation and in vitro functional assays resulting in high assay windows and high precision as well as an effective test method to distinguish agonists from antagonists among NPFF receptor ligands. The FP receptor binding assay offers a simple, rapid and low cost method for measuring ligand binding affinities that is amenable for HTS. The FP assay offers an additional advantage that does not involve radiolabeled compounds, remove the safety issues of handling radioactivity, and does not generate radioactive waste. Therefore, homogeneous FP receptor binding assays offer an alternative to radioligand binding assays and are the technology of choice for HTS of GPCRs.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

### Example 1

Materials

NPFF and other commercially available peptides were purchased from Bachem (Torrance, Calif.). Human recombinant NPFF2 receptor membrane preparation (receptor concentration=0.92-1.05 pmoles/mg protein) from stable CHO-K1 cells was obtained from Euroscreen s.a. (Brussels, Belgium). The amino terminal modified 5-carboxytetramethylrhodamine (TAMRA)-labeled NPFF (96% pure by high performance liquid chromatography monitored at 545 um) was obtained from PerkinElmer Life and Analytical Sciences (Boston, Mass.). Dyes, Chicago Sky Blue 6B and tartrazine, were purchased from Aldrich Chemical Co. (Milwaukee, Wis.). Protease inhibitor cocktail set III was purchased from Calbiochem-Novabiochem Co. (San Diego, Calif.). Prazosin, yohimbine, isoproterenol, alprenolol and propranolol were gifts from Professor Pan Dong Ryu at Seoul National University (Seoul, Korea). Simmondsin was a gift from Professor Kwan-Hwa Park at SNU. All other reagents were purchased from Sigma Chemical Co. (St. Louis, Mo.).

### Example 2

Instrumentation

Fluorescence polarization assays were performed using Wallac Victor²V™ (PerkinElmer Life and Analytical Sciences, Boston, Mass.). An excitation filter, 544(15) nm, and an emission filter, 595(60) nm, were factory supplied. Assays were conducted in Corning Costar® 384-well black polystyrene flat-bottomed plates (model 3654; Coming Inc., Acton, Mass.). Plate dimension tuning and calibration were conducted following the factory instrument setting manual. The signal integration time was 0.5 or 0.8 s/well to maximize Z′ value (Banks, P. et al., J. Biomol. Screen., (2002) 7, 111-117). The G-factor was set at 1.0 to give 20-35 mP with free TAMRA-NPFF (0.5 nM/well). The mP value for 0.5 nM TAMRA-NPFF alone in 50 mM HEPES buffer containing 0.1% Pluronic in the absence of receptor membrane was 77±3 mP. When G-factor varied from 0.75 to 1.25, mP values changed less than 10% from the mP value at G factor=1.0.

### Example 3

Receptor-ligand Binding Assays

Human NPFF2 receptor membrane homogenates were diluted in a binding buffer (50 mM HEPES buffer, pH 7.4 and various concentrations of detergents) to the membrane concentration required. In general, the binding assay was performed in a final volume of 40 μl: 20 μl of displacing ligand, 10 μl of fluorescent tracer, and 10 μl of diluted receptor membrane preparation (3.6-4.2 ug protein/well depending on the assay), which were added sequentially. The reaction was incubated at room temperature for 0.5-24 h before FP was measured depending on each experiment.

### Example 4

In vitro Functional Assays

Human NPFF2 receptor membrane homogenates were diluted in a binding buffer (50 mM HEPES buffer, pH 7.4 with 0.1% Pluronic F-127 containing 3 or 30 mM MgCl₂). The binding assay was performed in a final volume of 40 μl as follows: 10 μl of different concentrations of displacing ligand, 10 μl of tracer, and 10 μl of diluted receptor membrane preparation (3.6-4.2 ug protein/well), which were added sequentially. After 15 min, 10 μl of the binding buffer with 3-10 μM GDP and 5-50 μM GTPγS was added. The reaction was incubated at room temperature for 15-120 min.

### Example 5

Data Analysis

FP binding data were analyzed and fitted using GraphPad Prism® software (GraphPad Software, Inc., San Diego, Calif.). Competitive binding data was fit to a one-site competitive binding equation using nonlinear regression curve fitting. IC₅₀ values were determined by nonlinear regression analysis of the inhibition curves. Data points in the displacement curves and in the graphs of DMSO, nonionic detergents, Chicago Sky Blue 6B and tartrazine consist of an average of 4-6 repetitions. All error bars represent ±1 standard deviation unit of the spread of the data except for displacement curves, where the error bars represent ±SEM. mP is defined by the following equation: mP=1000×(I_{∥}−I⊥)/(I_{∥}+I⊥) where mP=milli-polarization, I_{∥}=parallel component of the emitted light, and I⊥=perpendicular component of the emitted light. Assay precision was assessed by calculating the Z′ value: Z′=1−[(3*σ_{bound})+(3*σ_{displaced})/|(mP_{bound}−mP_{displaced})|] where σ is the standard deviation in the signal, mP: the subscript “_{bound”} corresponds to the signal obtained in the absence of a displacing substance; and the subscript “_{displaced}” corresponds to the signal of a completely displaced ligand (Zhang, J-H. et al., J. Biomol. Screen., (1999) 4, 67-73; Banks, P. et al., J. Biomol. Screen., (2002) 7, 111-117). The denominator of the equation calculates the specific binding expressed in the absolute value of mP difference, and the numerator calculates the precision in both bound and displaced signals. Background correction was done after each analysis by subtracting blank parallel and perpendicular intensities from the respective intensities. According to Z′ value model, the Z′ values of above 0.5 indicate excellent assays.

Affinities (IC50 values) obtained for inhibition of FP binding were converted to Ki values using the Cheng-Prusoff equation (Cheng, Y.-C. et al., Biochem. Pharmacol., (1973) 22, 3099-3108) and the Kd value (0.23 nM) of NPFF2 receptor (batch 1168) was obtained by radioligand binding assay and provided by the membrane vendor. The concentration of [1251]DMe NPFF used was 0.008 nM.

### Example 6

Results

### Example 6.1

Assay Performance

In order to demonstrate the efficacy of FP in HTS assay for NPFF2 receptor, we have developed the NPFF2 receptor assay, which employs human recombinant neuropeptide FF2 membrane preparation, TAMRA NPFF, improved assay buffers and various displacing ligands on 384-well microplates. Table 1 summarizes the parameters that describe the performance of NPFF receptor assay employing three nonionic detergents. The two assays based on 0.1% Tween 20 and 0.1% Pluronic F-127 in 50 mM HEPES buffer, pH 7.39 achieved a performance suitable for screening based on Z′ values and the amount of membrane preparation expressing recombinant NPFF receptor equivalent to that used in the reported radioisotope filtration assays (Kotani, M. et al., Br. J. Pharmacol., (2001) 133, 138-144). NPFF₂ receptor expression with Bₘₐₓ of 0.92 pmol/mg and 0.5 nM of TAMRA-NPFF per well employed in the experiments, which used 0.1% Tween 20 and 0.1% Pluronic F-127 assay buffers, demonstrated satisfactory performance of Z′ values of above 0.50. However, 4% PEG in 50 mM HEPES buffer gave unacceptable Z′ values due to high standard deviations although the corrected FP assay window (ΔmP=the difference between the bound and displaced signals) was high with 125 mP. Attempts to improve the Z′ values by including EGTA, casein, calcium, magnesium or protease inhibitors in addition to 4% PEG in the HEPES buffer failed (data not shown). In general, the inclusion of protease inhibitors, metals and chelators appeared to lower the assay windows and precision when PEG- and Tween 20-containing buffers were used. Therefore, further optimization of HTS assays were carried out using a simpler assay buffer system with either 0.1% Tween 20 or 0.1% Pluronic F-127. The binding of TAMRA-NPFF (1.0, 1.6, 2.0, 2.6, and 4.0 nM) to 3.6 μg of human NPFF2 membrane protein was measured in the presence and absence of 1 μM NPFF. The optimized concentration (0.5 nM/well or 2.0 nM/vial) of TAMRA-NPFF was 2.2 fold higher than the K_{d} value obtained from the radioligand assay. Table 2 shows the Z′ value data obtained from five tracer concentrations investigated when 3.6 ug of NPFF receptor membrane protein and 0.1% Tween 20 in 50 mM HEPES buffer were used. It is evident that the tracer concentration of 2.0 nM (0.5 nM/well) gives Z′ values greater than 0.5, when monitored over 4 hours. There was only a narrow range of fluorophore concentration near to its K_{d} value that provided acceptable FP performance. High fluorophore concentration above 4 nM TAMRA-NPFF led to poor performance because of the presence of a large mole fraction of unbound fluorophore that acted to reduce the assay window.

TABLE 1
Assay Performance for TAMRA-NPFF Binding to Human Neuropeptide FF2 Receptor^{a}

| | Protein/ | Bound | | Displaced | σ^{c} in | | Assay |
|---|---|---|---|---|---|---|---|
| | well | signal | σ^{b} in Bound | Signal | Displaced | | window |
| Detergent | (μg) | (mP) | signal (mP) | (mP) | signal (mP) | Z′ value | corrected |
| 4% | 4.2 | 240 | 19 | 115 | 10 | 0.30 | 125 |
| Polyethylene | | | | | | | |
| glycol | | | | | | | |
| (PEG) | | | | | | | |
| 0.1% | 4.2 | 248 | 10 | 131 | 6 | 0.57 | 117 |
| Tween 20 | | | | | | | |
| 0.1% | 4.2 | 313 | 11 | 135 | 10 | 0.64 | 178 |
| Pluronic | | | | | | | |
| F-127 | | | | | | | |

^{a}Assay precision was used to calculate standard deviations and the Z′ values.
^{b}Intra-assay precision was determined by averaging six replicates of both bound and displaced signals.
^{c}Bound peptide was displaced fully using 1 μM neuropeptide FF.

TABLE 2
Impact of TAMRA-Labeled Neuropeptide FF Concentration
on Z′ values^{a}

| | TAMRA-NPFF Concentration | | | | |
|---|---|---|---|---|---|
| Time (min.) | 1.0 nM | 1.6 nM | 2.0 nM | 2.6 nM | 4.0 nM |
| 30 | 0.04 | 0.40 | 0.58 | 0.47 | 0.28 |
| 60 | 0.07 | 0.67 | 0.64 | 0.51 | −2.72 |
| 120 | −0.71 | 0.59 | 0.60 | 0.63 | −1.81 |
| 180 | −0.88 | 0.36 | 0.69 | −1.87 | −3.35 |
| 240 | 0.16 | 0.43 | 0.50 | −0.24 | 0.64 |

^{a}Z′ values were determined by averaging four replicates of bound and displaced signals from one 384-well plates. Bound peptide was displaced with 1 μM neuropeptide FF. The integration time was set at 0.8 s/well.

### Example 6.2

Effect of DMSO, Tween 20 and Pluronic F-127

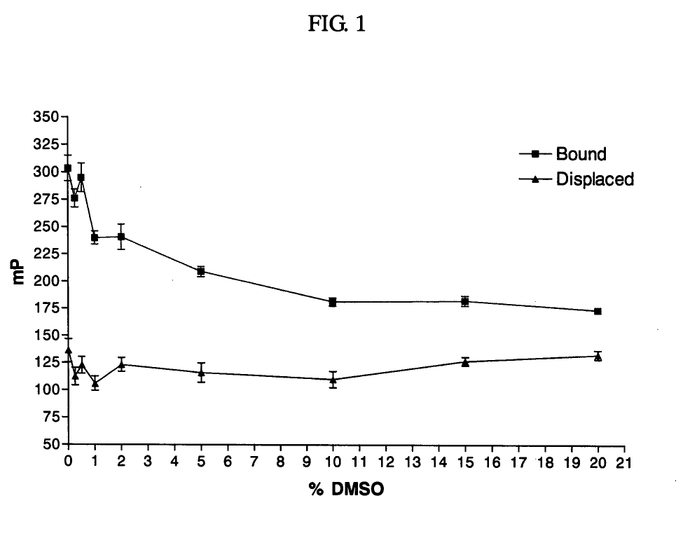

The sensitivity of FP assay windows to dimethyl sulfoxide (DMSO) has to be since most chemical libraries are stored in 100% DMSO. FIG. 1 demonstrates the effect of DMSO on TAMRA-labeled NPFF-human NPFF2 receptor binding. The assay tolerated up to 2% DMSO without significant loss of displaceable FP signal. The Z′ values for 0%, 0.25%, 0.5%, 1%, 2% and 5% DMSO ranged from 0.54 to 0.72. The inclusion of 5% DMSO lowered the FP assay window by 25 mP to 93 mP. Above 5% DMSO, bound and displaceable signals converged.

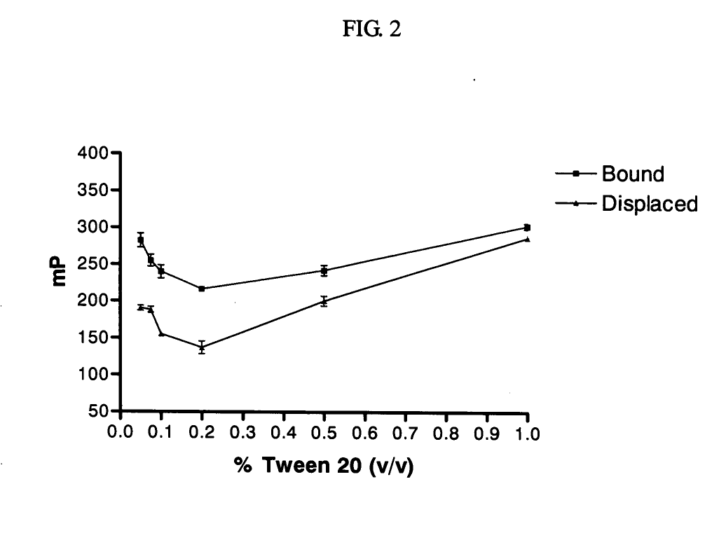

FIG. 2 shows the concentration effect of non-ionic detergent, Tween 20, on the tracer- NPFF2 receptor binding. The assay can withstand Tween concentrations up to 0.2% without any significant loss of assay windows. The Z′ values for 0.05%, 0.075%, 0.1% and 0.2% Tween 20 ranged from 0.57 to 0.60 when the reaction was incubated for one hour. Higher concentrations of Tween 20, 0.5% and 1.0%, significantly lowered the assay windows to less than 40 mP, and Z′ values of <0.5, an unacceptable HTS assay criteria (Zhang et al., J. Biomol. Screen. 4 (1999) 67-73; Banks et al., J. Biomol. Screen. 7 (2002) 111-117). 0.1% Tween 20 system gave the average assay window of 84 mP in this experiment and has been found later to be sensitive to the conditions of specific membrane preparations. For example, the assay window collapsed below 60 mPs even though the same batch of membrane preparations was used in the binding assays. Therefore, we have used 0.1% Tween 20 system to test the assay performance of new or stored membrane preparations due to its unique properties.

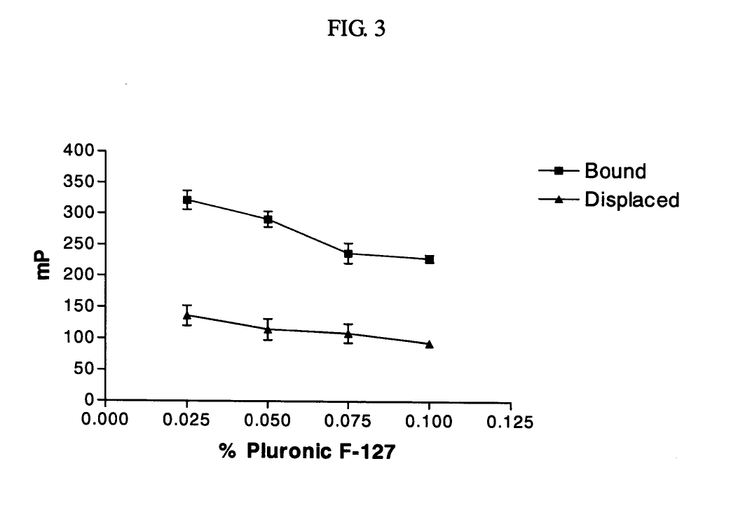

FIG. 3 depicts Pluronic F-127 titrations of the bound and displaced signals for TAMRA-labeled NPFF binding to human NPFF2 receptor. The assay can tolerate Pluronic concentration up to 0.1% without any significant effect on the displaceable FP signal. Among the four concentrations investigated, 0.1% Pluronic demonstrated the highest Z′ value of 0.75 after 90 min incubation. 0.1% Pluronic F-127 produced the FP assay window of 131-142 mP while the lower Pluronic concentrations gave even bigger assay windows. However, 0.025%, 0.05% and 0.75% Pluronic F-127 gave lower Z′ values of 0.50, 0.60 and 0.51, respectively. 0.1% Pluronic system has produced consistently high assay windows of >80 mPs and good Z′ values under various experimental conditions. Therefore, 0.1% Pluronic 127 system has been selected for the displacement curve and other binding studies.

### Example 6.3

Sensitivity to Colored Compounds

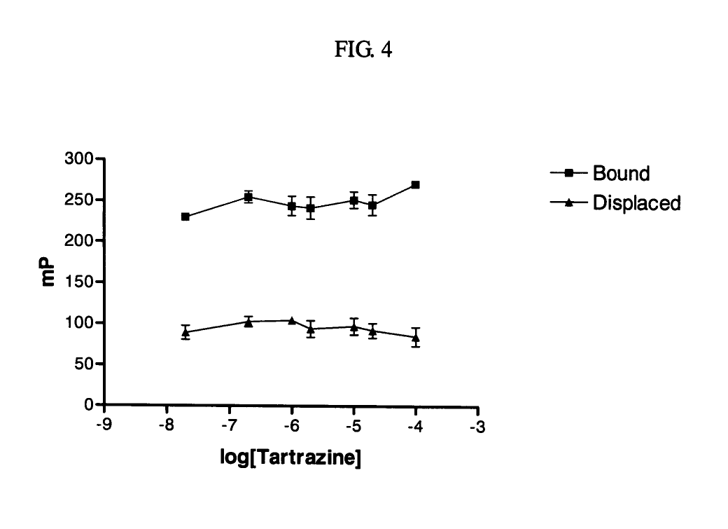

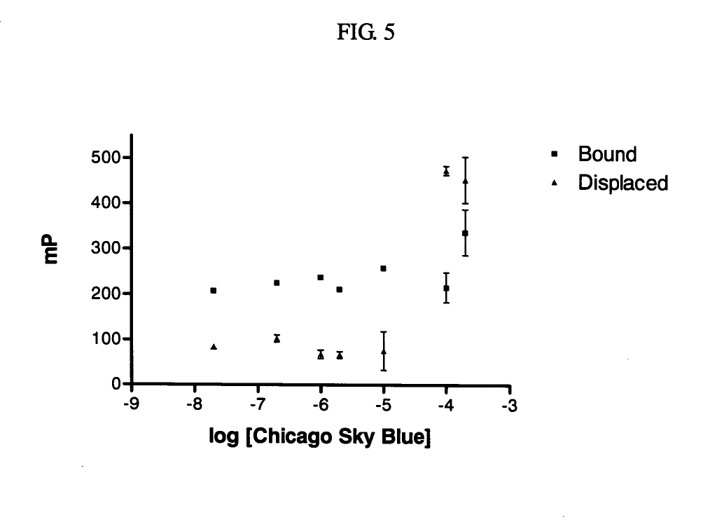

Compound libraries and some detergents such as Tween 20 contain various colors, which can lead to false-negative and false-positive HTS results. Two dyes were used to test the assay sensitivity to colored compounds. Tartrazine (λₑₓ=428 nm) is a yellow dye that contributes to fluorescence intensity and Chicago Sky Blue 6B (λₑₓ=620 nm) absorbs assay fluorescence (Banks, et al., J. Biomol. Screen., (2000) 5, 329-334). FIG. 4 demonstrates the sensitivity of FP assay to tartrazine. The assay trace in the presence of excess unlabeled NPFF displacer (1 μM final concentration) represents a hit in an agonist screen while the assay trace in the absence of excess unlabeled NPFF displacer represents a miss in the same screen. Comparing the two traces, up to 20 μM tartrazine may be used in the assay without any significant interference. Both bound and displaced signals are statistically equivalent to the controls where no tartrazine is present (mP_{bound}=263 mP and mP_{displaced}=120 mP in this experiment). From 100 μM, the bound signal appears to increase with increasing tartrazine concentration. This suggests that FP assays conducted with TAMRA-NPFF are much less susceptible to false-negative results in the presence of high levels of tartrazine. FIG. 5 shows the sensitivity of the assay to Chicago Sky Blue 6B. There is no significant effect of Chicago Sky Blue on the bound signal until its concentration of 2 μM is reached. Above 100 μM, the data are nonsensical since the displaced signals have polarization signals, which are 100 mP higher than the bound signals. The results indicate that FP assays conducted in the presence of a lot of Chicago Sky Blue or other blue colored compounds are prone to compound interference in the screening library.

### Example 6.4

Displacement Curves

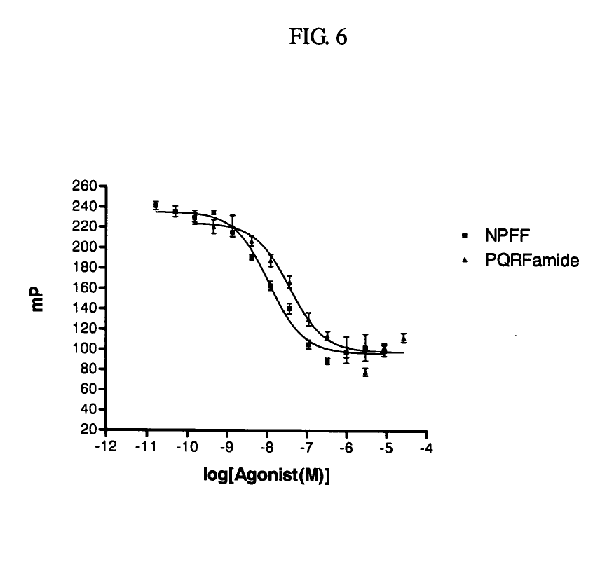

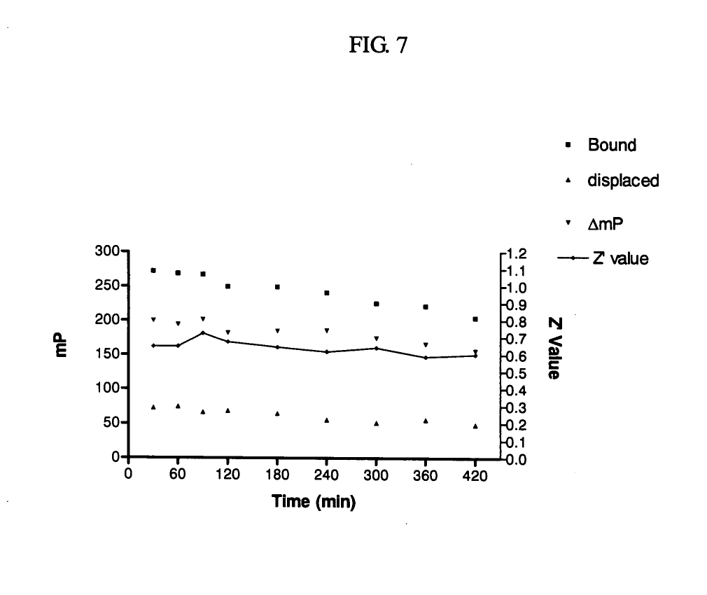

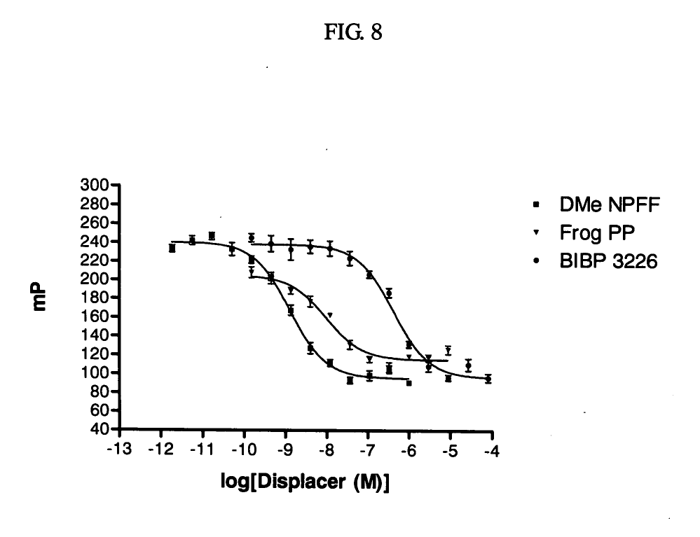

FIGS. 6 and 8 demonstrate examples of displacement curves for TAMRA-NPFF bound to human NPFF2 receptor by use of NPFF and other NPFF ligands. Table 3 presents the empirically determined Kᵢ and rank order of potency for all displacers used in the experiments, and lists the comparative Kᵢ and rank order of potency obtained by radioisotope filtration assays. For each of the agonists, FIG. 6 shows that a displaceable signal of over 80 mP is obtainable using the highest concentration of agonist. The high precision of current FP assay method is apparent from the error bars for each of the data points, and allows the assay method to be suitable for HTS. An amount of NPFF2 receptor membrane preparation that corresponds to 80% of radioisotope filtration assays yielded an excellent assay. However, increasing the membrane quantity two-fold (8.4 μg) or decreasing the quantity to one half (2.1 μg) yielded poor assays resulting in lower assay windows (data not shown). Peptides, NPFF, DMe FF, PQRFamide and frog PP, which share the common C-terminal amino acid sequence, were efficient in inhibiting TAMRA-NPFF binding with Kᵢ in nanomolar range. Time courses for binding reaction between TAMRA-NPFF and NPFF2 receptor are shown in FIG. 7. It is apparent that the equilibrium is achieved in approximately 30-60 min at room temperature to reach the maximum displaceable signal. The bound and displaced signals were stable for at least four hours resulting in excellent assay windows (181-201 mP in this example) when 0.1% Pluronic in 50 mM HEPES buffer, 1 μM DMe NPFF and 0.5 nM TAMRA-NPFF per well were employed. The precision of the assays remained stable with Z′ range of 0.58-0.64 though assay windows declined slowly between five and seven hours. However, the assay window collapsed to less than 50 mP and Z′ value was less than 0.3 when the plate was stored at room temperature overnight. Slow decline in bound signal over time (approximately 10 mP/hr) appears to be due to increased nonspecific binding of NPFF receptors to microplate surface through hydrophobic and ionic interactions.

The maximum mP values in Table 1 and FIG. 8 vary considerably. We observed a significant decline of maximum mP values and signal assay window (ΔmP) in both 0.1% Tween 20 and Pluronic as time goes. However, we were able to obtain good precision and acceptable assay windows above 80 mPs only in 0.1% Pluronic. The human NPFF2 receptor membrane vials received from the manufacturer at earlier months gave the highest mP values as shown in Table 1 while later shipments showed declines in bound signals as shown FIGS. 3, 7 and 8. There were also day-to-day mP variations of up to 20% as shown in Table 3. The solvents used in dissolving the displacers have significantly affected the bound and displaced signals. For example, we had to add 0.1% trifluoroacetic acid to the assay buffer to dissolve the relatively large frog PP consisting of 36 amino acids. This addition lowered the maximum mP value to 220 mP and increased the displaced signal to 120 mP, resulting in a narrower assay window of 100 mP (FIG. 8). The dissolved frog PP also showed a purple color contributing further to lower its assay window.

TABLE 3
Comparison of Inhibitory Potency

| | | [¹²⁵I]Ligand | [¹²⁵I]Ligand |
|---|---|---|---|
| | TAMRA-NPFF | Filtration | Filtration |
| | Human | Human | Human |
| Inhibitor | ^{a}Ki (nM) | ^{b}Ki (nM) | ^{c}Ki (nM) |
| [D-Tyr¹(NMe)Phe³] | 0.46 ± 0.05 | 3.2 ± 0.6 | 0.18 ± 0.04 |
| NPFF | | | |
| Frog PP | 5.2 ± 0.9 | 40 ± 11 | 7 ± 2 |
| NPFF | 3.24 ± 0.49 | ND | 0.21 ± 0.03 |
| PQRFamide | 13.2 ± 0.1 | 25 ± 3.0 | 6.8 ± 1.2 |
| BIBP3226 | 96 ± 13 | 1259 ± 111 | 84 ± 12 |

^{a}FP affinities, expressed as Kᵢ, were obtained from competition binding data using TAMRA-NPFF as a tracer and human NPFF2 receptor membrane preparation. Results are means ± S.E. obtained from three separate experiments.
^{b}Data from J. Biol. Chem. 275: 39324-39331 (2000); ND = not determined.
^{c}Data from Eur. J. Pharmacol. 451: 245-256 (2002).

### Example 6.5

Specificity

We have tested whether some known ligands of other receptors and an inhibitor of food intake interact with human NPFF2 receptors by use of the developed FP assay method with 0.1% Pluronic F-127. The assay windows of NPFF2 receptor with 10 μM each of bradykinin, capsaicin, prazosin, yobimbine, isoproterenol, epinephrine, propranolol, phenoxybenzamine, and simmondsin (Baek, J. S. et al., Biosci. Biotech. Biochem., (2003) 67, 532-539) ranged from −29 to 32 mP while the control assay window with 1 μM NPFF typically ranged from 99 to 123 mP (data not shown). Therefore, these compounds did not bind to NPFF2 receptor to any significant degree. It is interesting to observe that BIBP 3226 (Bonini, J. A. et al., J. Biol. Chem., (2000) 275, 39324-39331; Mollereau, C. et al., Eur. J. Pharmacol., (2002) 451, 245-256), a previously known neuropeptide Y1 antagonist and NPFF receptor antagonist, binds to NPFF2 receptor with low binding affinity of Ki of 96±13 nM as shown in FIG. 8 and Table 3.

### Example 6.6

Optimization Studies

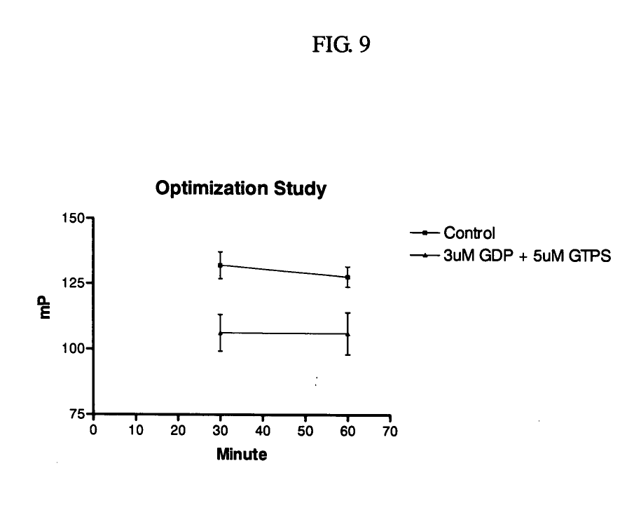

There was no significant difference in mP values whether 3 mM or 30 mM MgCl₂ was used with DMe NPFF as the agonist (data not shown). Thus, 3 mM MgCl₂ was used in the remaining GTP binding studies. Next we tested the effect of 3 uM and 10 uM GDP on the assay window with DMe NPFF in the presence of 3 mM MgCl₂. 3 uM GDP gave slightly higher assay windows (106 and 109 mP) than 10 uM GDP (96 and 103 mP) when monitored at 30 and 60 min. Therefore, 3 uM GDP was selected for the GTP binding studies. When 0.5 nM tracer and 1 uM DMe NPFF were incubated for 60 min, the bound signal observed was 226 mP. FIG. 9 shows that the displaced signal increased from 98 mP to 116 mP in the absence and presence of 5 μM GTPγS, resulting in the assay window of 128 and 109 mP, respectively. Thus, a net decline of 19 mP was observed in the presence of non-hydrolyzable GTP analog. The similar reduced assay windows were observed in the presence of 5 μM GTPγS when NPFF, PQRFamide or BIBP3226 was used as the displacer.

### Example 6.7

Functional Assays

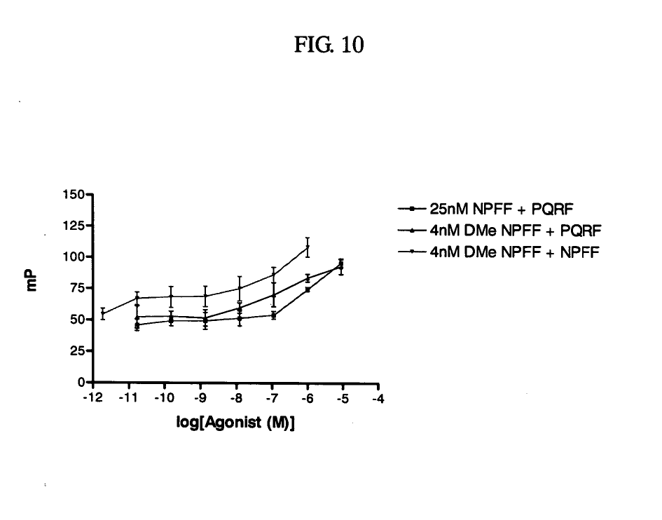

In order to distinguish agonists from antagonists, we have developed a cell membrane preparation-based GTP binding assay to monitor GPCR activation as an in vitro functional assay. Agonist binding to GPCRs stimulates the guanine nucleotide exchange. Thus, GDP bound to G_{α} subunit of G-proteins dissociates and is replaced by GTP through the cellular signal transduction mechanism (Devillers, J.-P. et al., Neuropharmacology, (1994) 33, 661-669; Payza, K. et al., J. Neurochem., (1993) 60, 1894-1899). In the in vitro functional experiments, the concentration of one agonist was fixed at 2˜3×IC50 value to give an assay window of 50-60 mP. The concentrations of the other agonist increased from 0.0019 nM to 9.0 μM per well. The data in FIG. 10 represent the observed signals after 30 min incubation upon the addition of 5 μM GTPγS solution. A combination of two agonists, either DMe NPFF+NPFF, DMe NPFF+PQRFamide, or NPFF+PQRFamide, induced a concentration-dependent signal increase (assay window) in the presence of 3 μM GDP and 5 μM GTPγS as shown in FIG. 10 (Agonist Effect). It is evident that these agonists of NPFF receptors showed biphasic affinity states in the presence of 3 uM GDP and 5 μM GTPγS.

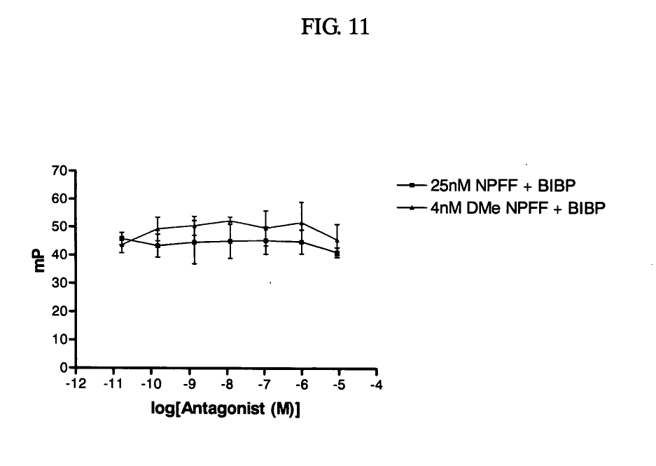

Next a combination of an agonist with a fixed concentration and an antagonist with varying concentrations was tested under the same conditions. FIG. 11 (Antagonist effect) shows that the combinations of an agonist, 25 nM NPFF or 4 nM DMe NPFF, and an antagonist, BIBP 3226, do not give the concentration-dependent signal increases. In fact, the presence of the antagonist appears to inhibit the signal increase at higher concentrations of the antagonist (0.1-9.0 μM). Therefore, the antagonist, BIBP 3226, gives a monophasic affinity state in in vitro functional assays.

All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the claims.

1. A method of determining whether an analyte binds to a target molecule in a sample comprising:
(1) contacting the target molecule with a tracer molecule, wherein a bond is formed between the target molecule and the tracer molecule to form a target molecule/tracer molecule complex;
(2) contacting the target molecule/tracer molecule complex with the sample containing the analyte; and
(3) measuring fluorescence polarization of the tracer molecule over time to determine whether the analyte binds to the target molecule, wherein decreased fluorescence polarization over time indicates that the analyte binds to the target molecule, and wherein the reaction is conducted in the presence of a surfactant, which is about 2.5-4:1 ethylene oxide and propylene oxide, respectively, having a critical micelle concentration of 0.01 to 0.1% wt.

2. The method according to claim 1, wherein the surfactant is about 10,000 to 14,000 daltons.

3. The method according to claim 1, wherein the surfactant is poloxamer 407 series or Pluronic® F-127.

4. A method of determining whether an analyte in a sample is an agonist for a receptor that is coupled to a second protein in which the second protein binds to the receptor when the receptor is activated, comprising:
(1) contacting the receptor with a tracer agonist and a fixed amount of a non-tracer known agonist in the presence of the second protein to form a mixture;
(2) contacting the mixture with the sample containing various concentrations of the analyte; and
(3) obtaining fluorescence polarization assay window of the tracer agonist at each concentration of the analyte over a set time, wherein increased assay window relative to increased concentration of the analyte indicates that the analyte is an agonist.

5. The method according to claim 4, wherein the contacting takes place in the presence of a surfactant, which is negative ionic surfactant, positive ionic surfactant, or non-ionic surfactant.

6. The method according to claim 5, wherein the surfactant is non-ionic.

7. The method according to claim 6, wherein the surfactant comprises about 2.5-4:1 ethylene oxide and propylene oxide, respectively, having a critical micelle concentration of about 0.01-0.1% wt.

8. The method according to claim 7, wherein the surfactant is poloxamer 407 series or Pluronic® F-127.

9. The method according to claim 4, wherein the receptor is G-protein coupled receptor.

10. The method according to claim 9, wherein the G-protein coupled receptor is adrenergic receptor, histamine receptor, muscarinic receptor, melanocortin receptor, neuropeptide FF receptor, epidermal growth factor receptor, neuropeptide Y receptor, dopamine receptor, cholecystokinin receptor, bombesin receptor, sphingosine 1-phosphate receptor, lysophosphatidic acid receptor, platelet-derived growth factor receptor, parathyroid hormone receptor, cannabinoid receptor, endothelin receptor, thrombin receptor, angiotensin receptor, somatostatin receptor, acetylcholine receptor, bradykinin receptor, vasopressin receptor, neurotensin receptor, or opioid receptor.

11. The method according to claim 10, wherein the G-protein coupled receptor is a neuropeptide receptor.

12. The method according to claim 11, wherein the neuropeptide receptor is NPFF receptor or NPFF2 receptor.

13. The method according to claim 9, wherein the tracer molecule comprises a fluorescent dye.

14. The method according to claim 4, wherein the known agonist is epinephrine, norepinephrine, histamine, alvameline, arecoline, cevimeline, milameline, sabcomeline, talsaclidine, tazomeline, xanomeline, melanotan-II oxotremorine, pilocarpine, arecoline, aceclidine, propoxy-TZTP, 3-Cl-propylthio-TZTP, hexylthio-TZTP, neuropeptide FF, ([D-Tyr¹, (N-Me)Phe³]NPFF), ([D-Tyr¹, D-Leu², D-Phe³] NPFF), xylazine, medetomidine, neuropeptide Y, dopamine, cholecystokinin, bombesin, sphingosine 1-phosphate, lysophosphatidic acid, parathyroid hormone, cannabinoid, endothelin, thrombin, antiotensin, somatostatin, acetylcholine, bradykinin, vasopressin, neurotensin, or opioid.

15. The method according to claim 4, wherein the tracer molecule is fluorescein-neuropeptide FF, BODIPY-TMR-neuropeptide FF, Texas Red-neuropeptide FF, 6-TAMRA-neuropeptide FF, X-rhodamine-neuropeptide FF, Cy3-neuropeptide FF, Cy5-neuropeptide FF, or TAMRA-neuropeptide FF.

16. The method according to claim 15, wherein the tracer molecule is TAMRA-neuropeptide FF.

17. The method according to claim 4, wherein the receptor is in the form of a membrane preparation.

18. The method according to claim 9, wherein a guanine nucleotide is added to the reaction.

19. The method according to claim 18, wherein the guanine nucleotide is guanosine 5′-diphosphate, guanosine 5′-[γ-thio] triphosphate, or guanosine 5′-[β, γ-imido] triphosphate.

20. A method of determining whether an analyte in a sample is an antagonist for a receptor that is coupled to a second protein in which the second protein binds to the receptor when the receptor is activated, comprising:
(1) contacting the receptor with a tracer agonist and a fixed amount of a non-tracer known agonist in the presence of the second protein to form a mixture;
(2) contacting the mixture with the sample containing various concentrations of the analyte; and
(3) obtaining fluorescence polarization assay window of the tracer agonist at each concentration of the analyte over a set time, wherein substantially constant assay window relative to increased concentration of analyte indicates that the analyte is an antagonist.

21. A composition for determining whether an analyte in a sample is an agonist or an antagonist for a receptor that is coupled to a second protein in which the second protein binds to the receptor when the receptor is activated, comprising: (i) receptor, membrane and second protein; (ii) tracer molecule; and (iii) surfactant.

22. The composition according to claim 21, wherein the receptor is G-protein coupled receptor.

23. The composition according to claim 22, wherein the second protein is G-protein.

24. The composition according to claim 23, comprising a guanine nucleotide.

25. A kit for determining whether an analyte in a sample is an agonist or an antagonist for a receptor that is coupled to a second protein in which the second protein binds to the receptor when the receptor is activated, comprising: (i) a container containing the receptor, membrane and second protein; (ii) a container containing tracer molecule.

26. The kit according to claim 25, wherein the kit comprises a guanine nucleotide.
